(19) United States
(12) Patent Application Publication
(10) Pub. No.: US 2011/0045471 A1
(43) Pub. Date: Feb. 24, 2011

(54) Methods for Assaying MC1R Variants and Mitochondrial Markers in Skin Samples

(75) Inventors: Ryan Parr, Thunder Bay (CA); Mark Birch-Machin, Tyne and Wear (GB); Andrew Harbottle, Tyne and Wear (GB); Robert Thayer, Thunder Bay (CA); Jennifer Creed, Thunder Bay (CA); Andrea Maggrah, Thunder Bay (CA); Kerry Robinson, Thunder Bay (CA); Gabriel Dakubo, Thunder Bay (CA); Brian Reguly, Vancouver (CA); Katrina Maki, Porcupine (CA)

Correspondence Address: MORGAN LEWIS & BOCKIUS LLP, 1111 PENNSYLVANIA AVENUE NW, WASHINGTON, DC, 20004, US

(73) Assignee: Genesis Genomics Inc., Thunder Bay, ON (CA)

(21) Appl. No.: 12/682,338

(22) PCT Filed: Sep. 14, 2008
(86) PCT No.: PCT/CA08/01801

§ 371 Date: Nov. 1, 2010

Related U.S. Application Data
(60) Provisional application No. 60/999,074, filed on Oct. 15, 2007.

(30) Foreign Application Priority Data
Oct. 11, 2007 (CA) PCT/CA2007/001790

(51) Int. Cl. C12Q 1/68

(52) U.S. Cl. 435/6

(57) ABSTRACT

The present invention relates to methods for predicting, diagnosing and monitoring skin states and skin diseases. The methods combine the use of non-invasive skin collecting techniques with one or more assays for determining mitochondrial DNA (mtDNA) aberrations and Melanocortin 1 Receptor (MC1R) variants, thereby providing a comprehensive tool for identifying, predicting and/or monitoring photoageing, ultraviolet radiation (UVR) damage or skin disease. The methods of the invention may also be effective in screening for new therapeutic agents, skin care products and treatment regimes, and may also be useful for monitoring the response of a subject to a preventative or therapeutic treatment.

### CROSS-REFERENCE TO RELATED APPLICATIONS

This application claims priority from PCT Application No. PCT/CA2007/001790, filed Oct. 11, 2007, and U.S. Application No. 60/999,074, filed Oct. 15, 2007, the entire contents of which are incorporated herein by reference.

### FIELD OF THE INVENTION

The present invention relates to methods for predicting, diagnosing and monitoring skin states and skin diseases. In particular, the present invention pertains to methods coupling non-invasive skin sampling techniques with assays for determining mitochondrial mutations and Melanocortin 1 Receptor (MC1R) variants for use as a comprehensive tool for predicting and monitoring disease, photoageing and ultraviolet radiation (UVR) damage. The methods are also useful for assessing the effectiveness of and response to therapeutic agents, skin care products and treatment regimes.

### BACKGROUND OF THE INVENTION

Skin disease represents a major health care challenge in today's world. With more than one million new cases of skin cancer diagnosed each year in the United States (National Cancer Institute, www.cancer.gov), predicting and diagnosing skin disease are important aspects of its management. Current diagnostic methods rely mainly on visible observations and biopsies. Detection methods that rely on visible observations, however, are not necessarily effective for diagnosing skin states or diseases, and do not detect risk or disease until after clinical manifestation. Furthermore, invasive methods such as biopsies, are not only traumatic for a subject being tested, they also increase the chances of infection. These methods must also be performed by a medical practitioner in order to be safely conducted, and typically do not provide an enriched sample of cells on the surface of skin, which are the cells generally involved in a reaction.

Non-invasive methods of diagnosing and monitoring skin states and diseases, therefore, represent important tools for patient management, and for assessing the efficacy of existing and new therapeutic agents, skin care products and skin care regimes. Furthermore, these methods may provide important information regarding the specific genetic changes underlying a subject's skin state, as well as their genetic predisposition for developing a skin disease. Identifying these genetic changes identifies potential drug targets and preventative measures, and may be critical in determining whether a person will actually respond to a particular therapeutic agent, skin care product or regime. As well, detection and diagnosis methods are important in assessing the safety of such therapies, products and measures.

Ultraviolet Radiation (UVR) Damage

Unknown or poorly quantifiable factors often contribute to skin damage as a result of ultraviolet radiation (UVR). Known factors include skin colour, frequency and severity of UVR exposure, melanin production, ratios of eumelanin to pheomelanin, and sunscreen use among others. Behavioural factors play a large role in the severity and level of damage to skin from UVR but are extremely difficult to assess clinically as they are dependent upon the accuracy of self-reporting and often a flawed awareness of a patient's sun lifestyle habits. Many individuals who use sunscreen regularly also use it improperly, failing to apply it in sufficient quantity or to reapply at the recommended intervals, creating a false sense of protection that can lead to increased exposure to UVR.

These factors among others necessitate the availability of a tool to closely monitor the success and appropriateness of the preventative measures and therapies being advised to prevent UVR damage to the skin, with consequences in the spectrum from premature photoaging to increasing skin cancer risk. In an effort to fully evaluate an individual's skin state, and as a result their risk of photoaging and skin cancer, it is imperative that health care practitioners are provided with as much insight as possible to understand and communicate their patient's risk factors. It would, therefore, be desirable to have a diagnostic method for assessing an individual's genetic predisposition to the damaging effects of UVR, as well as the identification of risk factors associated with an individual's particular sun lifestyle habits.

Such a method may include the collection of a DNA sample from the epidermal or dermal layers of an individual's skin for quantification of mitochondrial biomarkers indicative of UVR damage, as well as Melanocortin 1 Receptor (MC1R) genotyping, to identify variants involved in the control of melanogenesis. The combined use of both tests in tandem provide a comprehensive tool for health care providers to monitor, advise, and treat patients by considering both a patient's genetic predisposition to photoaging or cancer, and the result of the patient's ongoing exposure to ultraviolet radiation.

Mitochondrial Deletions Associated with UVR

Human skin tissue is highly complex and comprises numerous cell types. Accordingly, the identification of human skin cell biomarkers is of particular importance. To determine a reliable marker of cumulative UVR exposure in human skin, the inventors and others have examined the novel idea of using mitochondrial DNA (mtDNA), rather than nuclear DNA, as a biomarker of UV-induced DNA damage (Pang et al., 1994; Berneburg et al., 1997; Birch-Machin et al., 1998; Birch-Machin, 2000).

The use of mtDNA damage as a biomarker for cumulative sun-exposure in human skin is a relatively new field of research and previous work has simply compared mtDNA damage to distinguish between sun-protected and sun-exposed skin (Pang et al., 1994; Berneburg et al., 1997; Birch-Machin et al., 1998). This approach is limited because non-melanoma skin cancer (NMSC) is predominantly formed on body sites which are “usually” exposed to the sun when outdoors as opposed to sites that are “occasionally” exposed to the sun (Armstrong, 2004).

In the present Applicant's co-pending PCT application bearing publication no. WO/06/111029 (the contents of which are incorporated herein by reference), a 3895 by deletion in human mitochondrial DNA (mtDNA) was identified as a biomarker of UV-induced DNA damage. This deletion was identified in the minor arc spanning nucleotides 547-4443. This deletion had previously been associated with Kearns Sayre Syndrome and Chronic Progressive External Opthalmoplegia (Moraes et al., 1995).

Examples in PCT. publication no. WO/06/111029 demonstrate that that the frequency of occurrence of the 3895 by mtDNA deletion is significantly different between body sites that are “usually” versus “occasionally” exposed to the sun. In addition, the examples demonstrated a link between the etiology of the 3895 by deletion and the UVR component of sunlight by inducing the 3895 by deletion in vitro with repetitive sub-lethal doses of a UVA+UVB light source.

Importantly, skin samples analysed in WO/06/111029 were not assessed with regard to the individual's genetic predisposition to sun damage and risk for skin cancer. In addition, these samples were obtained by painful methods of skin collection previously known in the art.

Melanocortin 1 Receptor (MC1R) Genotyping

The melanocortin-1 receptor gene (MC1R) encodes a membrane-bound receptor protein that is central to melanin synthesis. The coding region of MC1R is highly polymorphic and associations of variants with pigmentation phenotypes and risk for melanoma and non-melanoma skin cancer have been reported (Rees, Pigment Cell Research, Vol. 13(3), 135-140(6), June 2000; Kanetsky et al. Cancer Epidemiology Biomarkers & Prevention Vol. 13, 808-819, May 2004). The incidence rate of melanoma is greatest in fair-skinned, sun sensitive individuals, suggesting that the ability to respond to UV exposure, by increased synthesis of melanin is an important factor in melanoma defense. Family studies have shown that individuals with fair skin and red hair harbor functionally significant changes in both MC1R alleles. The alleles D84E, R142H, R151C, R160H and D294H have high penetrance for red hair and fair skin. Two lower penetrance alleles (V60L, V92M) are also common factors in melanoma risk.

MC1R is, therefore, a major determinant of sun sensitivity and a genetic risk factor for skin cancer. Assessing DNA or amino acid sequence of the Melanocortin 1 Receptor (MC1R) gene to identify whether certain variants which are associated with increased sun sensitivity, susceptibility to DNA damage and increased skin cancer risk are present provides a valuable tool to identify patients with greater risk who are in need of more aggressive monitoring and treatment measures. Evaluation of phenotypic characteristics associated with sun sensitivity alone would not be able to elucidate this increased risk.

Skin Sampling

Current methods for the collection of skin samples for use in the diagnosis or characterization of diseases, such as skin cancer, include invasive or painful methods that can cause substantial discomfort to the individual being tested. Examples of current methods for the collection of skin samples include punch biopsy, tapelift, and surgical excision. In addition to the discomfort caused by the current methods for skin collection, these methods must also be performed by a medical practitioner in order to be safely conducted.

Such invasive techniques have other disadvantages including risk of infection, inconvenience of sample collection, and the possibility that collected samples can be lost or misidentified. Infection and inconvenience are magnified in situations in which frequent or regular sample collection is required, such as with UVR exposure monitoring regimes and the assessment of long-term therapy. Further, the time and cost associated with these invasive test methods make it difficult to rapidly genotype and assess DNA damage for large populations of individuals. It would, therefore, be desirable to provide a non-invasive or minimally invasive skin collection methodology that may be conducted easily and rapidly in a home, clinical or cosmetic setting.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide methods for assaying MC1R variants and mitochondrial markers in skin samples. In accordance with an aspect of the present invention, there is provided a diagnostic method for determining the skin state and genetic predisposition of a subject to UVR damage, comprising:
(a) collecting tissue samples from a subject;
(b) assaying a first tissue sample for mitochondrial DNA (mtDNA) aberrations;
(c) assaying a second tissue sample for one or more melanocortin 1 receptor (MC1R) variants; and
(d) determining the skin state and genetic predisposition of the subject to UVR damage based on the detection of the mtDNA aberrations and MC1R variant(s) in the skin samples.

In accordance with another aspect of the invention there is provided use of the method of the invention for predicting photoaging, UVR damage or skin disease.

In accordance with another aspect of the invention there is provided use of the method of the invention for determining a prophylactic or therapeutic treatment for preventing or ameliorating photoaging, UVR damage or skin cancer.

In accordance with another aspect of the invention there is provided a non-invasive method for monitoring photoaging, UVR damage or skin disease, comprising:
(a) collecting a skin sample from a subject using a non-invasive skin sampling technique;
(b) assaying the skin sample for mitochondrial DNA (mtDNA) aberrations at regular intervals over a prescribed period of time; and
(c) determining any changes in mtDNA aberrations identified over the prescribed period of time.

In accordance with another aspect of the invention there is provided a method for monitoring a subject's response to a preventative or therapeutic treatment for photoaging, UVR damage or skin disease, comprising:
(a) collecting a first skin sample from a subject;
(b) assaying the first skin sample for mitochondrial DNA (mtDNA) aberrations;
(c) assaying the first skin sample for one or more melanocortin 1 receptor (MC1R) variants;
(d) determining the skin state and genetic predisposition of the subject to UVR damage based on the detection of the mtDNA aberrations and MC1R variant(s) in the first skin sample;
(e) providing a preventative or therapeutic treatment for photoaging, UVR damage or skin disease;
(f) collecting a second skin sample from a subject;
(g) assaying the second skin sample for mitochondrial DNA (mtDNA) aberrations;
(h) repeating steps (f) and (g) at regular intervals over a prescribed period of time; and
(i) comparing the level of mtDNA aberrations between the first skin sample and the skin samples taken at regular intervals to detect changes in mtDNA aberrations, thereby monitoring the effectiveness of the treatment; and
(j) optionally, adjusting the treatment based on the genetic predisposition of the subject and the detected changes in mtDNA aberrations.

In accordance with another aspect of the invention there is provided a method of screening for an effective therapeutic or cosmeceutic agent for the treatment of photoaging, UVR damage or skin disease, comprising;
(a) collecting a first skin sample from a subject;
(b) assaying the first skin sample for mitochondrial DNA (mtDNA) aberrations;
(c) treating the subject with the therapeutic or cosmeceutic agent;
(d) collecting a second skin sample from a subject following a prescribed period of time;
(e) assaying the second skin sample for mitochondrial DNA (mtDNA) aberrations; and
(f) comparing the level of mtDNA aberrations between the first skin sample and the second skin sample against a control to determine the effectiveness of the therapeutic or comesceutical agent.

In accordance with another aspect of the invention there is provided a method for determining the level of photodamage of a subject, comprising:
(a) collecting a skin sample from a subject;
(b) assaying the skin sample for mitochondrial DNA (mtDNA) deletions;
(c) comparing the level of mtDNA deletions of the skin sample against a population of mtDNA deletions categorized according to age co-horts, and assigning a photoage to the subject; and
(d) determining the level of photodamage of the subject by comparing the subject's chronological age to the assigned photoage.

In accordance with another aspect of the invention there is provided a diagnostic kit for determining the skin state and genetic predisposition of a subject to UVR damage, comprising:
(a) material for collecting tissue samples; and
(b) suitable primers, probes and reagents for carrying out MC1R genotyping and the detection of mtDNA aberrations.

### BRIEF DESCRIPTION OF THE FIGURES

These and other features of the invention will become more apparent in the following detailed description in which reference is made to the appended drawings.

FIG. 1 shows real-time PCR data relating to the 3895 by mtDNA deletion levels in skin samples collected from the nose and the heel using the method of the present invention.

FIG. 2 shows real-time PCR data relating to levels of the 3895 by mtDNA deletion in skin cells collected from various body sites using the method of the present invention.

FIG. 3 shows real-time PCR data relating to levels of the 3895 by mtDNA deletion in skin cells collected from various body sites using the method of the present invention.

FIG. 4 is a gel showing the presence of amplification products present in samples collected from various non-invasive skin collection methods.

FIG. 5 is a gel showing the presence of amplification products present in samples collected from various non-invasive skin collection methods.

FIGS. 6 to 11 show genotyping results for subjects tested for allele variants.

FIG. 12 shows a gel providing results of an assay testing for the R160H allele variant.

FIG. 13 shows a graph depicting the level of 3895 by mtDNA deletion of a population categorized into age cohorts.

### DETAILED DESCRIPTION OF THE INVENTION

The present invention provides methods of predicting, diagnosing and monitoring skin states and skin diseases. The methods comprise coupling non-invasive skin sampling techniques with assays that determine mitochondrial mutations and Melanocortin 1 Receptor (MC1R) variants. In this regard, the methods provide a comprehensive tool for assessing genetic predisposition and risk factors associated with disease, ageing and ultraviolet radiation (UVR) damage. The methods also allow for the assessment of a patient's response to therapeutic agents, skin care products and treatment regimes.

Definitions

Unless defined otherwise, all technical and scientific terms used herein have the same meaning as commonly understood by one of ordinary skill in the art to which this invention belongs.

As used herein, the term “about” refers to approximately a +/−10% variation from the stated value. It is to be understood that such a variation is always included in any given value provided herein, whether or not it is specifically referred to.

As used herein, “alleles” means one of several alternative forms of a given DNA sequence occupying a specific place on a chromosome.

As used herein, “cycle threshold” (C_{T}) is the point at which target amplification of a nucleic acid sequence rises above background, as indicated by a signal such as a fluorescence signal. The C_{T} is inversely related to the quantity of the sequence being investigated.

As used herein, “diagnostic” or “diagnosing” means using the presence or absence of a mutation or combination of mutations as a factor in disease diagnosis or management. The detection of the mutation(s) can be a step in the diagnosis of a disease.

As used herein, “deletions” means removal of a region of DNA or mtDNA from a contiguous sequence of a nucleic acid. Deletions can range in size from one base to thousands of bases or larger.

As used herein, “mitochondrial DNA” or “mtDNA” is DNA present in mitochondria.

As used herein, “mutation” encompasses any modification or change in nucleic or mitochondrial DNA from the wild type sequence, including without limitation point mutations, transitions, insertions, transversions, translocations, deletions, inversions, duplications, recombinations or combinations thereof. The modification or change of the sequence can extend from a single base change to the addition or elimination of an entire DNA fragment.

The term “sample” refers to any preparation derived from skin of a subject. For example, a sample of cells obtained using the non-invasive method described herein can be used to isolate polynucleotides, polypeptides, or mitochondrial DNA, for the methods of the present invention. Samples for the present invention, typically are taken from the dermis or epidermis of the skin. The samples are preferably taken of the skin surface using non-invasive or minimally invasive skin sampling methods discussed herein.

The term “skin” refers to the outer protective covering of the body, consisting of the corium and the epidermis, and is understood to include sweat and sebaceous glands, as well as hair follicle structures. In one embodiment, the skin is mammalian skin, preferably human.

As used herein the term “skin state” refers to the condition of the skin with respect to the amount of UVR damage accumulated due to sun exposure, tanning beds or UVR associated diseases and the like.

The terms “therapy” and “treatment,” as used interchangeably herein, refer to an intervention performed with the intention of improving a subject's status. The improvement can be subjective or objective and is related to ameliorating the symptoms associated with, preventing the development of, or altering the pathology of a skin disease, disorder or state. Thus, the terms therapy and treatment are used in the broadest sense, and include the prevention (prophylaxis), moderation, reduction, and curing of a disease, disorder or state, at various stages. Preventing deterioration of a subject's status is also encompassed by the term. Subjects in need of therapy/treatment thus include those already having the disease, disorder or state as well as those prone to, or at risk of developing, the disease, disorder or state and those in whom the disease, disorder or state is to be prevented.

Assays for Determining Skin State and Assessing Genetic Predisposition to Skin Disease

The methods of the present invention comprise coupling non-invasive skin sampling techniques with assays effective in identifying an individual's genetic predisposition to skin disease and risk factors associated with UVR exposure. The assays may be conducted alone or in combination with one another. The combined use of these assays provides a unique ability to simultaneously assess genetic risk factors and sun lifestyle habits, thereby providing a diagnostic tool for health care professionals to better monitor, advise and treat patients that are prone to or suffering from photoaging, UVR damage or a skin disease.

Assay for Detection of Mitochondrial Mutations

Mitochondrial DNA (MtDNA) dynamics are an important diagnostic tool. Mutations in mtDNA are often preliminary indicators of developing disease and may act as biomarkers indicative of risk factors associated with disease onset. As well, as a result of the higher copy number per cell of mitochondrial DNA as compared to nuclear DNA it is a more robust target for assays relying on extremely minute quantities of nucleic acids. The methods of the present invention, therefore, couple non-invasive techniques for collecting skin samples with an assay for detecting mutations in human mitochondrial genome.

As discussed herein, measuring the level of mitochondrial DNA aberrations in a skin sample can identify the skin state of a patient with respect to cumulative sun exposure and UVR damage. Furthermore, measurement of mtDNA at regular intervals such as biweekly or monthly can provide health care professionals with a real-time, quantitative monitoring tool for comparison against treatment recommendations in order to determine their effectiveness in preventing skin damage caused by UVR.

The present invention, therefore, provides a method for determining the skin state of a patient with respect to cumulative UVR damage, comprising combining a non-invasive skin collecting technique with an assay for detecting a mitochondrial aberration. In accordance with one embodiment of the invention, the mitochondrial mutation is selected from the group consisting of deletions, substitutions, and insertions. In accordance with another embodiment of the invention, the mutation is an mtDNA deletion. In accordance with yet another embodiment of the invention the mutation is the 3895 by mtDNA deletion identified in PCT application no. WO/06/111029. In accordance with still another embodiment of the invention, the mutation is the 3895 by deletion as set forth in SEQ ID NO:1. In accordance with still a further embodiment of the invention, the mtDNA mutation corresponds to the sequence as set forth in SEQ ID NO:2.

Exemplary methods for non-invasively collecting skin samples and assaying mitochondrial mutation are provided in the Examples section. Extraction of mtDNA from a sample may be undertaken using any suitable known method. MtDNA extraction is followed by amplification of all or a region of the mitochondrial genome, and may include sequencing of the mitochondrial genome, as is known in the art and described, for example, in Current Protocols in Molecular Biology (Ausubel et al., John Wiley & Sons, New York, 2007). Likewise, methods for detecting the presence of mutations in the mtDNA can be selected from suitable techniques known to those skilled in the art. For example, analyzing mtDNA can comprise sequencing the mtDNA, amplifying mtDNA by PCR, Southern, Northern, Western South-Western blot hybridizations, denaturing HPLC, hybridization to microarrays, biochips or gene chips, molecular marker analysis, biosensors, melting temperature profiling or a combination of any of the above.

Any suitable means to sequence mitochondrial DNA may be used. Preferably, mtDNA is amplified by PCR prior to sequencing. The method of PCR is well known in the art and may be performed as described in Mullis and Faloona, 1987, Methods Enzymol., 155: 335. PCR products can be sequenced directly or cloned into a vector which is then placed into a bacterial host. Examples of DNA sequencing methods are found in Brumley, R. L. Jr. and Smith, L. M., 1991, Rapid DNA sequencing by horizontal ultrathin gel electrophoresis, Nucleic Acids Res. 19:4121-4126 and Luckey, J. A., et al, 1993, High speed DNA sequencing by capillary gel electrophoresis, Methods Enzymol. 218: 154-172. The combined use of PCR and sequencing of mtDNA is described in Hopgood, R., et al, 1992, Strategies for automated sequencing of human mtDNA directly from PCR products, Biotechniques 13:82-92 and Tanaka, M. et al, 1996, Automated sequencing of mtDNA, Methods Enzymol. 264: 407-421.

Melanocortin 1 Receptor (MC1R) Genotyping

The melanocortin 1 receptor is a key control point in melanogenesis and determines the amount of pigment accumulation in the skin. Given that skin pigment determines the degree of an individual's natural protection to carcinogenic UVR, MC1R genotyping can determine susceptibility to DNA damage caused by UVR exposure and assess risk factors associated with skin cancer. As such, evaluating the DNA or amino acid sequence of the Melanocortin 1 Receptor (MC1R) gene to identify whether certain variants which are associated with increased sun sensitivity and susceptibility to UVR damage, provides a valuable tool for identify individuals at greater risk for disease and who may be in need of more aggressive monitoring and treatment measures.

The methods of the present invention couple non-invasive skin collecting techniques with an assay for MC1R genotyping. DNA or RNA extracted from skin samples may be used to identify one or more MC1R variant alleles. For example, the seven allele variants D84E, R142H, R151C, R160H, D294H, V60L, and V92M associated with increased melanoma risk may be tested simultaneously. Alternatively, MC1R genotyping for the five allelic variants most commonly associated with increased risk of skin cancer, i.e. D84E, R151C, D294H, V60L, and V92M may be undertaken. The MC1R gene, including variants, is set forth in SEQ ID NO: 3. In addition, the MC1R RNA sequence is set forth in SEQ ID NO: 4.

The present invention, therefore, provides a method for determining the genetic predisposition of an individual to skin damage or cancer comprising combining a non-invasive skin collecting technique with an assay for identifying the presence of one or more MC1R variants in a skin sample. In accordance with one embodiment of the invention, the MC1R variants tested are selected from the group consisting of allelic variants D84E, R142H, R151C, R160H, D294H, V60L, and V92M. In accordance with another embodiment of the invention, the MC1R variants tested are selected from the group consisting of D84E, R151C, D294H, V60L, and V92M.

Exemplary methods for non-invasively collecting skin samples and performing MC1R genotyping are provided in the Example section. One of skill in the art will appreciate that, unlike the detection of mtDNA mutations, which may fluctuate through the lifetime of an individual as a result of UVR exposure patterns, the results from MC1R genotyping will not change throughout an individual's life. Accordingly, MC1R may only be tested once during the lifetime of a patient, if so desired.

Genotyping assays such as those used for evaluating the MC1R variants will produce identical results in an individual regardless of which body tissue is samples as the sequence of the MC1R gene is inherited and does not change throughout ones lifetime. An individual skilled in the art would recognize that though the sample can be acquired from the non-invasive collection method for skin it may also be collected from any other body tissue to obtain the same genotype. Collection of suitable tissues for use in the methods of the invention would be well understood in the art and may include such nonlimiting examples as buccal tissue, muscle tissue, nerver tissue and the like. The same is not true for the mitochondrial assays which are tissue-specific somatic mutations. Thus, in accordance with one embodiment of the invention, there is provided a method comprising collecting a tissue sample from a subject, wherein the tissue sample is other than a skin sample. In accordance with another embodiment of the invention, there is provided a method comprises collecting a tissue from a subject, wherein the tissue is a buccal sample.

Extraction of DNA or RNA from a sample may be undertaken using any suitable known method. Detection of specific alleles may be determined using such art recognized techniques as ABI's a qPCR Taqman SNP genotyping assay (https://products.appliedbiosystems.com/ab/en/US/direct/). Likewise, methods of preparing customized primers and probes are well known in the art (see, for example, Ausubel, et al., Current Protocols in Molecular Biology, John Wiley & Sons, Inc., NY).

Alternatively, allele variant assessment may be based on evaluating the amino acid sequence of the MC1R gene. Techniques for undertaking amino acid analysis are well understood in the art and may include, for example, high performance liquid chromatography (HPLC).

Determining Skin Type and Risk of Skin Cancer

Not only can the methods of the invention be used to assess an individual for the presence of allelic variants, but may also be utilized to determine a skin type that is an approximation of the Fitzpatrick phototype of the individual. In this regard, the results of MC1R genotyping additionally allows for the grouping of individuals into specific skin types characterized by the associated risk for cancer.

These skin types are defined below:

Skin Type 1 is characterized in that the individual has two or more of the four allelic variants most associated with risk of skin cancer.

Skin Type 2 is characterized in that the individual has one of the four allelic variants most associated with risk of skin cancer.

Skin Type 3 or higher is characterized in that the individual has none of the four allelic variants most associated with risk of skin cancer.

Thus, in accordance with one embodiment, there is provided a method of identifying an individual's skin type and skin cancer risk factor on the basis of MC1R genotyping.

Non-Invasive Methods of Collecting Skin Tissues

The present invention provides for non-invasive or minimally invasive techniques of collecting skin samples for genotyping or diagnostic tests. In the context of the present invention, “minimally invasive” refers to those techniques that result in the penetration of one or more layers of the skin, but draw little or no blood. Non-limiting examples of non-invasive or minimally-invasive techniques used for collecting skin samples include, but are not limited to, tapelift using surgical tape, Sterile swab wetted with 8% mandelic acid, Sterile swab wetted with distilled water, wax strip, cotton tip swap, scraping of skin using a sterile surgical blade, scraping of skin using a wooden scraper, sticky surface of an adhesive pad (CapSure™ Clean-up Pad, Arcturus), film from LCM MacroCap™ (Arcturus), heated film from LCM MacroCap™ (Arcturus) and employing a small gauge needle (for example, 28 gauge), to collect micro-cores of skin tissue.

The sample may be collected from the dermal or epidermal layer of the skin and may be derived from such areas of the body as, for example, the heel, nose, inner arm, ear, mouth, scalp, chest, shoulder, buttock, back, face, nape of the neck, hand and/or head. The sample can be used either directly as obtained from the source or following a pre-treatment to modify the character of the sample. Thus, the skin sample can be pre-treated prior to use, for example, with preservatives, reagents, and the like.

One skilled in the art will understand that more than one sampling technique may be employed at a single time. Furthermore, where a course of collections are required, for example, for the monitoring of a skin state over time, the same or different techniques may be used alone or together throughout the test period. In this regard, skin collections may be taken once only, or at regular intervals such as biweekly or monthly.

One of skill will also appreciate that certain collection methods yield extremely low levels of nucleic acids (approximately 0.1 ng) which would not be useful for an assay targeting nuclear DNA; however, mitochondrial DNA targets that are in much greater abundance (approximately 1000 fold greater) would be uniquely suited to a collection method with such extremely low yields. The examples below show that skin cells collected via the non-invasive methods of the present invention provide sufficient mtDNA for obtaining results comparable to mtDNA obtained via previously used skin collection methodologies.

With reference to specific methods of the invention, in one embodiment there is provided a non-invasive collection technique which involves the use of a sterile swab, such as those used in the collection of buccal cells or cotton-tip swabs. The sterile swab is removed from its packaging and is rubbed on a skin site of interest. Preferably, the site is swabbed approximately 15 times in order to ensure that a sufficient number of skin cells are collected for genotyping or diagnostic purposes. Although the present invention is described below with reference to a specific example, the method may also be used to collect skin samples for the diagnosis or characterization of disease, aging, or exposure to ultraviolet radiation, and the identification of mutations associated therewith.

Following the swabbing of the skin, the swab is deposited into a sterile tube. Buffer may be added to the tube as necessary in order to maintain the integrity of the genetic material (i.e. DNA) contained therein. The DNA is then extracted utilizing well known methods in the art.

In another embodiment of the present invention, a minimally invasive technique which employs a very small gauge needle (28 or 29 gauge) is used to collect skin cells for the purpose of genetic investigation. In this embodiment, skin cells are collected from the dermis and epidermis of a subject by piercing through a tented layer of the skin such that little or no blood is drawn, but a microscopic amount of dermal and epidermal tissue is adhered to the inner core of the needle. The skin may be tented by raising the skin using, for example, fingers, tweezers, or other forms of clamp. The skin material is contained in the needle until it is extracted for further processing (ie. DNA extraction). To express the skin sample from the needle, phosphate buffered saline is deposited into the column of the needle and then forced through with the plunger into a sterile tube. DNA is extracted utilizing well known methods in the art. As illustrated by example below, this minimally invasive method for the collection of a skin sample yields sufficient DNA or mtDNA for the assessment of DNA or mtDNA damage, for example, caused by UV radiation. As with the previous embodiment, this method of obtaining skin samples is safe and painless. Further, as illustrated below, allows for sufficient DNA or mtDNA to be collected for conducting accurate assays.

Diagnosing and Monitoring Skin States and Detecting Genetic Risk Factors Associated with Disease

As described herein, the assays of the invention may be used alone, for example to assess skin state or genetic risk, or in combination to evaluate both genetic predisposition to skin disease and identification of risk factors associated with an individual's particular lifestyle. As discussed below, the identification and monitoring of these factors are important in the determination of effective preventative and treatment measures against UVR damage, photoaging and skin disease.

Diagnosing and Monitoring Skin States

Many individuals who use skin care products such as sunscreens and sunblocks regularly use the products improperly, failing to apply them in sufficient quantity or to reapply at the recommended intervals, creating a false sense of protection that can lead to increased exposure to UVR. Furthermore, until more recently, sunscreens and sunblocks regularly applied by individuals during sun exposure generally protected against the mutagenizing effects of UVB, but failed to contain agents directed at the harmful effects of UVA radiation.

These factors among others necessitate the availability of a tool to determine both initial skin state with respect to UVR damage and, through a course of studies, monitor the success and appropriateness of preventative measures and therapies being advised to prevent further UVR damage to the skin. Measuring the level of mitochondrial DNA deletions in the skin of a patient by non-invasively collecting skin samples at regular intervals such as biweekly or monthly (or any other suitable interval) can provide health care professionals with a real-time, quantitative monitoring tool to compare against treatment recommendations to determine their effectiveness in preventing skin damage caused by UVR. This collection methodology permits regular sampling without any side effect or discomfort and can be performed at home, if desired. Surprisingly, techniques for collecting skin samples such as swabbing are uniquely suited to the detection of mtDNA mutations, as the extremely low yields of DNA provided by swabbing are suffice for undertaking such analysis.

Turning now to the examples, in one embodiment of the invention the method is used to collect skin cells for the quantification of biomarkers associated with damage caused by UV radiation (see Example 1). Specifically, the method of the present invention was used to collect skin samples for testing for deletions in the human mitochondrial genome, namely the 3895 by mtDNA deletion described above. The example shows that skin cells collected via the non-invasive method of the present invention provides sufficient mtDNA for obtaining results comparable to mtDNA obtained via previous skin collection methodologies.

One of ordinary skill in the art will understand that the non-invasive collection of DNA samples from the epidermal or dermal layers of human skin for quantification of a mitochondrial DNA target provides a means for a health care worker to monitor the effectiveness of treatment regimes and lifestyle recommendations for the prevention of UVR associated skin and DNA damage with the intent to prevent such consequences as photoaging and skin cancer.

One of ordinary skill will also appreciate the utility of mtDNA analysis for use by health care providers in identifying poor sun habits or ineffective protection methods. Such utility is demonstrated in Example 6, where according to one embodiment of the invention, the results of sunscreen use was analysed for two individuals of similar age and skin tone to assess the effectiveness of sun protection regimes.

Determination of Photoage

An additional diagnostic method of the invention that makes use of a minimally invasive sampling procedure coupled with the identification of a mitochondrial marker is the determination of photoage. As described in the Example section, the purpose of this method is to evaluate an individual's UVR exposure by sampling the individual's skin tissue on the face or head using a minimally invasive skin collecting technique and measuring the amount of a mitochondrial target associated with UVR damage such as mtDNA deletion. This result is then compared against a database of results achieved by the same method and the individual is classified into an age cohort based upon the level of mtDNA deletion detected. For assignment of a photoage, the results of an individual's mtDNA analysis is compared against their chronological age to determine if they have higher or lower photodamage than other individuals in their age group.

Thus, by utilizing minimally invasive techniques to collect micro-cores of skin tissue for analysis of mitochondrial DNA markers, health care provides can identify an individual's photoage and determine whether their photoage is consistent or inconsistent with their chronological age. Determination of photoage thereby provides a means for health care professionals to more appropriately prescribe preventative and treatment measures to combat photoaging, UVR damage or skin disease.

Determining Genetic Predisposition to Disease

In order to fully evaluate an individual's risk of photoaging and their risk of skin cancer it is imperative that health care providers are provided with as much information as possible to understand and communicate their patient's risk factors. The utilization of MC1R genotyping not only contributes to an individual's susceptibility to DNA damage caused by UVR exposure and risk of developing skin cancer, it provides a valuable tool to identify patients with greater risk who are potentially in need of more aggressive monitoring and treatment measures.

The present invention therefore provides a non-invasive method for the collection of DNA samples for MC1R genotyping. In accordance with one embodiment of the invention, DNA extracted from cells collected by the method of the present invention may be used to identify one or more allele variants associated with increased melanoma risk. The method may be used to assess an individual for the presence of these variants and to additionally determine a skin type of the individual. The results of MC1R genotyping therefore allows for the grouping of individuals into specific skin types characterized by the associated risk for cancer.

Coupled Analysis

The combined use of MC1R genotyping and mtDNA analysis provides a tool for health care professionals to monitor, advise and better treat patients by considering both genetic predisposition and the end results of their sun lifestyle habits. This combined use of assays provides a unique ability to simultaneously assess risk factors as well as consequences. Importantly the MC1R test may be temporally removed from repeated testing with the non-invasive collection for mtDNA testing and still be used in tandem. As discussed above, this is possible because the information obtained from the MC1R test will not change throughout an individual's life though the DNA damage levels elucidated by measuring mitochondrial DNA deletion will fluctuate as a result of UVR exposure patterns.

Noteworthy is the fact that skin damage caused by UVR is a multifactorial process. Accordingly, it cannot be assumed that an individual with MC1R variants will always have higher damage than an individual without, nor will it be the case that an individual who reports regular sunscreen use will necessarily have lower damage than one who reports no sunscreen use. Thus, the coupling of MC1R genotyping with mtDNA analysis provides health care workers. with the unique insight they require to make more informed recommendations to those in their care.

Evaluation of Therapeutic Agents and Skin Care Products

The method of the present invention may also be used for widespread skin screening for both medical and cosmeceutical purposes. The method of the present invention may be used for genotyping and/or to measure various biomarkers associated with skin cancer (both non-melanoma skin cancer and melanoma). The ability to assess the level of DNA damage in an individual's skin due to UV radiation at any time point and from any external anatomical location provides the foundation for a unique and informative screening test for skin health and to assess the safety and efficacy of existing and new therapeutic agents, skin care products and skin care regimes. Furthermore, by identifying the specific genetic changes underlying a subject's skin disease or state, it may be readily determined whether and to what extent a patient will respond to a particular therapeutic agent, skin care product or regime.

Kits

The collection materials used in the method of the present invention may be packaged, depending on the desired application, into a consumer kit or a medical kit to be used in a clinical environment. Such kits could not only include one or more sampling means, such as sterile swabs, cotton tip swabs or needles, but other materials necessary for genotyping and/or the identification of mtDNA mutations.

The kits can optionally include reagents required to conduct a diagnostic assay, such as buffers, salts, detection reagents, and the like. Other components, such as buffers and solutions for the isolation and/or treatment of a test sample, may also be included in the kit. One or more of the components of the kit may be lyophilised and the kit may further comprise reagents suitable for the reconstitution of the lyophilised components.

Where appropriate, the kit may also contain reaction vessels, mixing vessels and other components that facilitate the preparation of the test sample. The kit may also optionally include instructions for use, which may be provided in paper form or in computer-readable form, such as a disc, CD, DVD or the like.

To gain a better understanding of the invention described herein, the following examples are set forth. It will be understood that these examples are intended to describe illustrative embodiments of the invention and are not intended to limit the scope of the invention in any way.

### Examples

### Example 1

### Analysis of 3895 by Human mtDNA Deletion

The method of the present invention was used to analyze the 3895 by mtDNA deletion identified in PCT application no. WO/06/111029. Collection and extraction of the mtDNA was conducted as provided below.

1. Skin samples were collected by swabbing a skin site approximately 15 times with a sterile swab. Skin samples were collected from heel (n=41), nose (n=43), inner arm (n=20), ear (n=5), shoulder (n=5), buttock (n=5), and back (n=5).

2. mtDNA was extracted using a commercially available kit (QiaAMP™ DNA Micro Kit, product no. 56304, Qiagen, Maryland USA) according to the manufacturer's protocol.

3. Double stranded DNA was quantified using the HS-DNA Quant-it™ dsDNA HS Assay Kit (product no. Q32851, Invitrogen, California USA) on the Qubit™ Fluorometer (product no. Q32857), Invitrogen, California USA).

4. The level of the 3895 by deletion was then quantified by real-time PCR (rt-PCR) using the iQ Sybr Green Supermix™ (product no. 170-8882, Bio-Rad, California USA) and the following primers:

(SEQ ID NO: 5)

| | |
|---|---|
| Forward | 5′-CTGCTAACCCCATACCCCGAAAATGTTG-3′; |

(SEQ ID NO: 6)

| | |
|---|---|
| Reverse | 5′-GAAGGATTATGGATGCGGTTGCTTGCGTGAG-3′. |

In this example, the pair of amplification primers are used to amplify a target region indicative of the presence of the 3895 by deletion. The forward primer overlaps a spliced region of mtDNA after deletion of the 3895 by sequence has occurred (ie. a splice at a position between 547 and 4443 of the mtDNA genome). Therefore, extension of the overlapping primer to create the correct size amplification product can only occur if the 3895 by section is deleted.

In the step of quantifying the 3895 by deletion, the RT-PCR reaction was set up as follows:
12.5 ul of iQ Sybr Green Supermix™;
350 nmol forward primer (SEQ ID NO: 5);
350 nmol reverse primer (SEQ ID NO: 6);
5 ul of template (approximately 0.5 ng dsDNA);
water to 25 ul;

Cycling Parameters:
Step 1. 95° C. for 3 minutes;
Step 2. 95° C. for 30 seconds;
Step 3. 67.5° C. for 30 seconds;
Step 4. 72° C. for 30 seconds;
Step 5. Plate Read
   45 cycles of steps 2-5
   Melting Curve 55-110° C. reading every 3 seconds at 1° C. intervals
   Hold at 10° C. for 10 minutes.

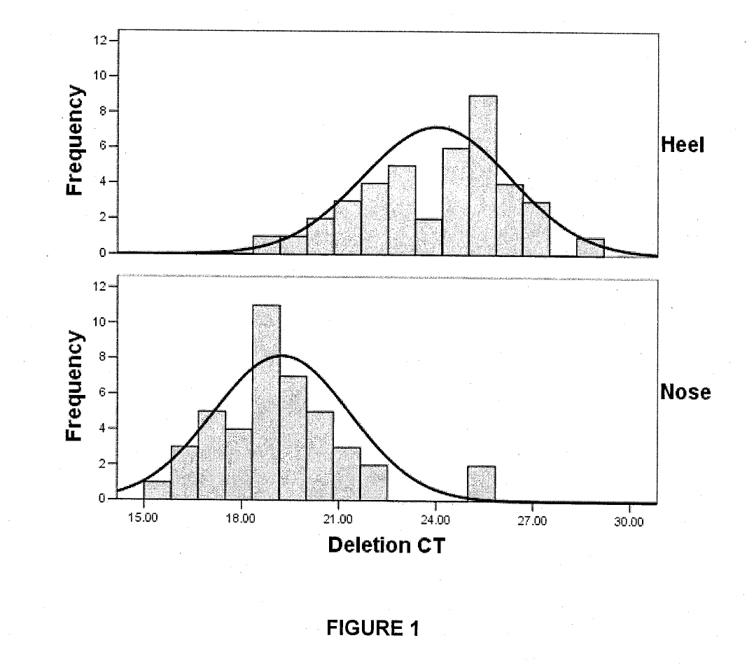

The results of these assays are shown in FIGS. 1 to 3 and demonstrate a clear distinction between skin swabs taken from areas rarely exposed to sunlight/UV radiation (ie. heel and buttocks) and those usually exposed (ie. nose and ear). Levels of the 3895 by deletion are significantly elevated in areas receiving a higher level of UV radiation such as the nose and shoulder when compared to areas generally protected from UV radiation such as the heel and the buttocks.

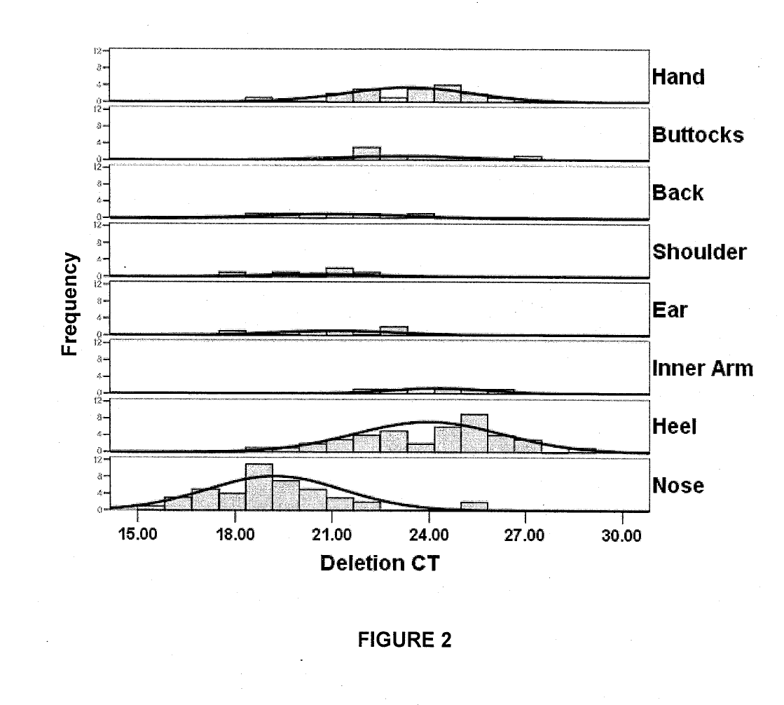

As shown in FIGS. 1 and 2, the real time PCR cycle thresholds (C_{T}) for the 3895 by deletion indicate that there is a higher incidence of the deletion in skin sites usually (nose or ear) or occasionally (shoulder or back) exposed to UV radiation compared to those sites that are rarely exposed (heel or buttocks).

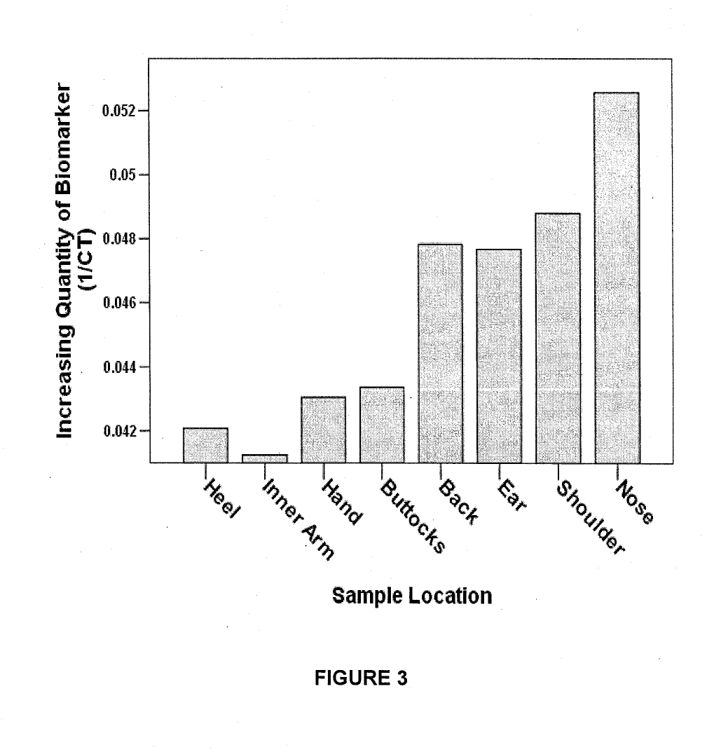

FIG. 3 shows that mtDNA collected from skin cells obtained from sites that are usually exposed to UV radiation (e.g. nose or ears) are characterized by increased levels of the 3895 by deletion marker than mtDNA collected from skin cells obtained from sites rarely exposed to UV radiation (e.g., heel or inner arm).

These results also show the effectiveness of collecting skin samples in accordance with the present invention, in order to obtain sufficient mtDNA to conduct the assays. As such, the non-invasive skin collection methods of the present invention are similarly effective for obtaining mtDNA for analysis as invasive methodologies, for example, the methods used in the Applicant's PCT publication no. WO/06/111029.

### Example 2

### Comparison of Skin Collection Methods

Five different non-invasive skin collection methodologies were tested in order to identify which, if any, would yield sufficient quantity and quality of nucleic acids for molecular analyses such as quantitative real-time PCR. The five methods tested were:
Tapelift using surgical tape;
Biore® adhesive strip;
Sterile swab wetted with 8% mandelic acid;
Sterile swab wetted with distilled water; and
Wax strip.

The tapelift, Biore strip and wax strip were applied to the surface of the skin following the application of 70% isopropanol to sterilize the area. Firm pressure was applied and then the tape or strip was removed quickly. The swabs were first deposited in a sterile solution of either 8% mandelic acid, or distilled water and then rubbed firmly on the skin site of interest after the skin had been cleaned with 70% isopropanol.

Following the collection of skin cells from 3 individuals each collection medium was deposited into 200 ul phosphate buffered saline solution (PBS) and incubated overnight at 56° C.

All of the samples were then subjected to nucleic acid extraction using the Qiagen's QiaAMP™ DNA Mini Kit, buccal swab protocol (product no. 51304). The purified samples were quantified using the NanoDrop™ ND-1000 Spectrophotometer to determine if the extraction procedure was successful.

TABLE 1
Determination of Quantity of DNA Extracted from Skin Collected
by Five Non-invasive Methods

| | DNA | | DNA Purity |
|---|---|---|---|
| | concentration | | 260:280 |
| Sample ID | ng/uL | A260 nm | Ratio |
| 1 biore | −0.42 | −0.008 | 0.48 |
| 1 swab with water | 5.03 | 0.101 | 1.6 |
| 1 swab with mandelic acid | 5.05 | 0.101 | 1.74 |
| 1 surgical tape | 2.22 | 0.044 | 4.4 |
| 1 wax | 3.52 | 0.07 | 1.69 |
| 2 biore | −0.02 | 0 | −0.09 |
| 2 swab with water | 2.74 | 0.055 | 1.97 |
| 2 swab with mandelic acid | 4.9 | 0.098 | 1.82 |
| 2 surgical tape | 2.99 | 0.06 | 1.78 |
| 2 wax | 2.17 | 0.043 | 1.84 |
| 3 biore | −0.26 | −0.005 | 0.24 |
| 3 swab with water | 3.26 | 0.065 | 3.15 |
| 3 swab with mandelic acid | 2.71 | 0.054 | 12.1 |
| 3 surgical tape | 2.45 | 0.049 | 4.26 |
| 3 wax | 2.67 | 0.053 | 4.25 |

When considering both nucleic acid concentration as well as the purity of the sample, the most consistent results were achieved for the swab samples using either water or mandelic acid as a wetting agent, or the wax samples.

Next, a PCR was performed on all samples to determine if amplification inhibitors were present or significant degradation of the sample had occurred during processing. Samples were amplified according to the following conditions:

TABLE 2
PCR Conditions for Sample Amplification

| Reagent | Final Concentration in Reaction |
|---|---|
| 10X reaction Buffer | 1X |
| dNTPs | 0.4 mM each |
| BSA | 1X |
| 12s primer (forward) | 0.4 uM |
| 12s primer (reverse) | 0.4 uM |
| Taq LA (Takara p/n RR002B | 1.25 Units |
| Template | 5 ul (of above concentration) |
| Water | To 25 ul |

The primers used were mitochondrial DNA primers having the sequences provided below:

| | |
|---|---|
| 12s primer sequence forward | |
| 5′-CGTTCCAGTGAGTTCACCCTC-3′ | (SEQ ID NO: 7) |
| 12s primer sequence reverse R | |
| 5′-CACTCTTTACGCCGGCTTCTATT-3′ | (SEQ ID NO: 8) |

The amplification reactions were cycled on a DNA Engine Tetrad (Bio-Rad) according to the following protocol:

1. 94° C. for 2 minutes

2. 94° C. for 30 seconds

3. 64° C. for 30 seconds

4. 72° C. for 30 seconds

5. Repeat steps 2-4 39 times

6. 4° C. HOLD

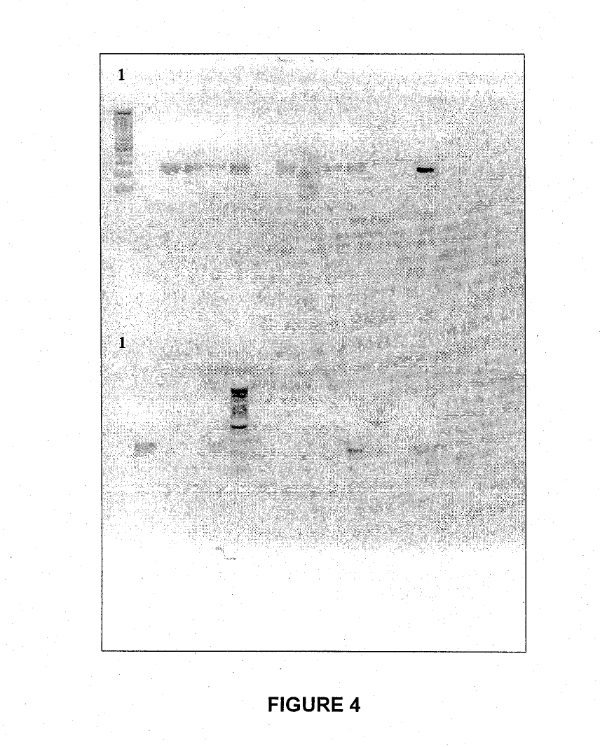

Amplification products were then electrophoresed on a 2% agarose gel and stained with ethidium bromide. The amplification results are provided in FIG. 4, where the top half of the gel contains:

Lane 1 500 ng 100 bp GeneRuler SM0323 (Fermentas)

Lane 2 Biore from Individual 1

Lane 3 Swab with water from Individual 1

Lane 4 Swab with mandelic acid from Individual 1

Lane 5 Surgical tape from Individual 1

Lane 6 Wax from Individual 1

Lane 7 Biore from Individual 2

Lane 8 Swab with water from Individual 2

Lane 9 Swab with mandelic acid from Individual 2

Lane 10 Surgical tape from Individual 2

Lane 11 Wax from Individual 2

Lane 12 empty

Lane 13 Negative amplification control

Lane 14 Positive amplification control

Lanes 15-18 empty

And where the bottom half of the gel contains:

Lane 1 Biore from Individual 3

Lane 2 Swab with water from Individual 3

Lane 3 Swab with mandelic acid from Individual 3

Lane 4 Surgical tape from Individual 3

Lane 5 Wax from Individual 3

Lane 6 500 ng 100 bp GeneRuler SM0323 (Fermentas)

Lane 7 Biore extract negative control

Lane 8 Swab with water extract negative control

Lane 9 Swab with mandelic acid extract negative control

Lane 10 Surgical tape extract negative control

Lane 11 Wax extract negative control

Lane 12 empty

Lane 13 Negative amplification control (duplicate loading to Lane 13 above)

Lane 14 Positive amplification control (duplicate loading to Lane 14 above)

Lane 15-18 empty

Results

No mtDNA was amplified from mtDNA collected from skin cells harvested using the Biore strips. The swabs for both the water and the mandelic acid amplified, though the water swab amplified more brightly. The surgical tape amplified sporadically. The wax amplified brightly however the extract negative control in Lane 11 of the bottom half of the gel was contaminated likely as a result of the non-sterile nature or handling difficulties associated with the wax.

With all factors considered this example demonstrated that the use of a sterile swab is the preferred method of collection of a non-invasive skin sample. The swab can be dry or wetted with various liquids to facilitate collection or buffering of the sample.

### Example 3

### Comparison of Additional Skin Collection Methods

In this example five additional methods for the non-invasive collection of skin samples were tested in order to identify which, if any, would yield sufficient quantity and quality of nucleic acids for molecular analyses such as quantitative real-time PCR.

From a single individual, skin samples were collected twice using the following methods:
scraping of skin using a sterile surgical blade
scraping of skin using a wooden scraper
sticky surface of an adhesive pad (CapSure™ Clean-up Pad, Arcturus)
film from LCM MacroCap™ (Arcturus)
heated film from LCM MacroCap™ (Arcturus)

The skin was first prepared by cleansing with a 70% isopropanol wipe. The wooden scraper and the surgical blade were passed firmly over the skin surface to remove skin cells and then deposited into a centrifuge tube. The adhesive pad and films were pressed firmly against the skin without rubbing to collect skin cells.

The multiple collections were processed using two different nucleic acid extraction methods. The first set was extracted using a proteinase K digestion as is well known in the art while the second set was extracted using the QiaAMP DNA Mini Kit (Qiagen 51304).

The samples processed with the Qiagen kit were then quantified using the NanoDrop ND.-1000 Spectrophotometer. Those in the PK digestion set were not as they were not cleaned up enough to facilitate this type of quantification.

TABLE 3
Determination of Quantity of DNA Extracted from Skin Collected
by Further Collection Methods (Qiagen extracted DNA)

| | DNA | | DNA Purity |
|---|---|---|---|
| | concentration | | 260:280 |
| Sample ID | ng/uL | A260 nm | Ratio |
| AE Buffer | −1.09 | −0.022 | 1.6 |
| Woodscrape | 2.06 | 0.041 | 0.95 |
| Capsure | 5.59 | 0.112 | 1.29 |
| Blade | 2.32 | 0.046 | 1.41 |
| Blade −ve control | 1.19 | 0.024 | 0.8 |
| Capsure −ve control | 2.47 | 0.049 | 1.2 |
| Woodscrape −ve control | 2.92 | 0.058 | 1.35 |
| QIaGEN kIT −ve control | 2.4 | 0.048 | 1.83 |

Samples were amplified according to the protocol provided in example 2.

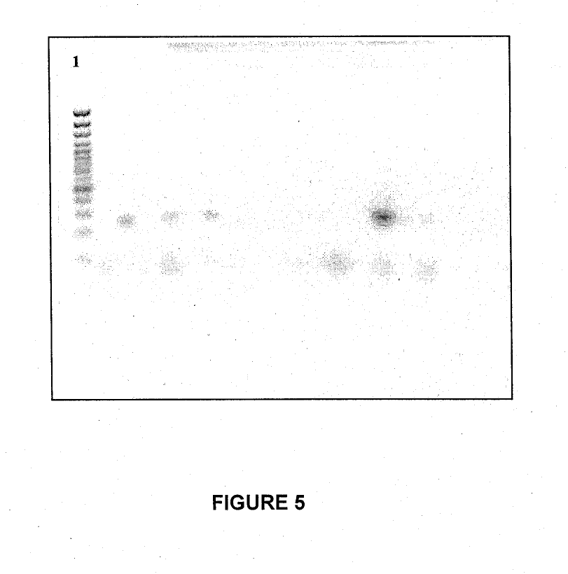

Amplification products were then electrophoresed on a 2% agarose gel and stained with ethidium bromide. Results are shown in FIG. 5, where the gel contains:

Lane 1 500 ng of 100 bp GeneRuler (SM0323)

Lane 2 Negative Amplification control

Lane 3 Positive amplification control

Lane 4 PK buffer wood scrape

Lane 5 PK buffer surgical blade scrape

Lane 6 PK buffer CapSure pad

Lane 7 PK buffer MacroCap

Lane 8 PK buffer MacroCap heated

Lane 9 PK buffer wood scrape negative extraction control

Lane 10 PK buffer surgical blade scrape negative extraction control

Lane 11 PK buffer CapSure negative extraction control

Lane 12 PK buffer MacroCap negative extraction control

Lane 13 PK buffer MacroCap heated negative extraction control

Lane 14 QiaAMP surgical blade scrape

Lane 15 QiaAMP CapSure pad

Lane 16 QiaAMP wood scrape

Lane 17 QiaAMP surgical blade scrape negative extraction control

Lane 18 QiaAMP CapSure pad negative extraction control

Lane 19 QiaAMP wood scrape negative extraction control

Lane 20 QiaAMP reagent negative control

The surgical blade scrape and the MacroCap™ were amplified using the PK buffer, while the CapSure™ pad amplified well using the QiaAMP™ kit. When compared to skin swabbing, the amount of mtDNA collected and the amount of amplified product obtained using the methods tested in this example were not found to be as effective.

### Example 4

### Collection of Skin Samples Using Needle

Needles were used to collect skin samples from 5 different body sites of 9 individuals. The body sites included the eyebrow, earlobe, nape of the neck, hand, and heel.

Using a needle as described above, the skin was pinched or tented between the thumb and forefinger of the sample collector's hand. The needle was passed through the skin, drawing little or no blood. The skin sample was extracted from the needle by depositing phosphate buffered saline into the column of the needle and then forcing the sample from the needle with a plunger into a sterile tube. DNA was then extracted from this volume containing the skin tissue using the QiaAMP™ DNA Mini Kit™ (Qiagen product no. 51304).

The samples were then amplified in order to identify the 3895 bp mtDNA deletion. The reaction conditions and cycle parameters for this example were the same as for example 1 provided above. The results are presented in Table 3.

TABLE 4
Results for Skin Samples collected via Needle

| | Cycle |
|---|---|
| | Threshold |
| Sample | C(t) |
| Subject 1 left eyebrow | 25.82 |
| Subject 1 left earlobe | 25.98 |
| Subject 1 neck | 26.26 |
| Subject 1 right hand | 26.73 |
| Subject 1 right heel | 27.93 |
| Subject 2 left eyebrow | 30.85 |
| Subject 2 left earlobe | 25.6 |
| Subject 2 neck | 23.92 |
| Subject 2 right hand | 27.01 |
| Subject 2 right heel | 35.55 |
| Subject 3 left eyebrow | 29.64 |
| Subject 3 left earlobe | 23.51 |
| Subject 3 neck | 24.52 |
| Subject 3 right hand | 22.47 |
| Subject 4 left eyebrow | 24.23 |
| Subject 4 left earlobe | 23.64 |
| Subject 4 neck | 24.96 |
| Subject 4 right hand | 25.93 |
| Subject 4 right heel | 23.58 |
| Subject 5 left eyebrow | 22.35 |
| Subject 5 left earlobe | 24.39 |
| Subject 5 neck | 22.06 |
| Subject 5 right hand | 22.04 |
| Subject 5 right heel | 22.6 |
| Subject 6 left eyebrow | 22.15 |
| Subject 6 left earlobe | 19.87 |
| Subject 6 neck | 25.87 |
| Subject 6 right hand | 27.91 |
| Subject 7 left eyebrow | 29.89 |
| Subject 7 left earlobe | 19.18 |
| Subject 7 neck | 25.49 |
| Subject 7 right hand | 23.41 |
| Subject 7 right heel | 27.05 |
| Subject 8 left eyebrow | 21.82 |
| Subject 8 left earlobe | 21.32 |
| Subject 8 neck | 23.51 |
| Subject 8 right hand | 16.35 |
| Subject 8 right heel | 20.57 |
| Subject 9 left eyebrow | 25.09 |
| Subject 9 left earlobe | 26.76 |
| Subject 9 neck | 27.74 |
| Subject 9 right hand | 25.24 |
| Subject 9 right heel | 22.39 |

It is clear that the material obtained through this collection method is sufficient for molecular analyses such as real-time PCR. Specifically, the amplification product indicative of the 3895 by mtDNA deletion has been detected and quantified as evidenced by Table 3. Therefore, the collection of skin samples via the needle collection method yields sufficient DNA for use in an assay of this kind.

### Example 5

### Detection of MC1R Variants

Buccal samples were collected from three subjects. Each subject rinsed their mouth with tap or bottled water and expelled the water. A cotton swab was removed from its protective packaging and grasped by the handle. While holding the handle of the cotton swab, the cotton tip was placed within the subject's mouth and the cotton tip was rubbed on the inside of the cheeks for 30 seconds. The swabs were placed in sterile tubes and sealed for further processing.

Detection of specific alleles was determined using ABI's a qPCR Taqman SNP genotyping assay. Each subject was tested for the seven allele variants D84E, R142H, R151C, R160H, D294H, V60L, and V92M.

The genotypes for D84E, R142H, R151C, V60L, and V92M were identified using ABI assays (https://products.appliedbiosystems.com/ab/en/US/direct/) as follows:

V60L—Assay #C751905410

D84E—Assay #C751905520

V92M—Assay #C203340520

R142H—Assay #C2754163410

R151C—Assay #C203340420

Customized primers and probes, as listed below, were used for the detection of the D294H allele variants.

| | |
|---|---|
| Forward Primer MC1R-294: | |
| CGCCCTCATCATCTGCAATG; | SEQ ID NO: 9 |
| Reverse Primer MC1R-294: | |
| GGCTGTGGAAGGCGTAGAT; | SEQ ID NO: 10; |
| Probe 1 MC1R-294V1 VIC: | |
| CCATCATCGACCCCCT; | SEQ ID NO: 11 |
| Probe 2 MC1R-294M1 FAM: | |
| CCATCATCCACCCCCT. | SEQ ID NO: 12 |

The reactions conditions used for all assays were as outlined in the Custom Taqman™ SNP Genotyping Assays Protocol.

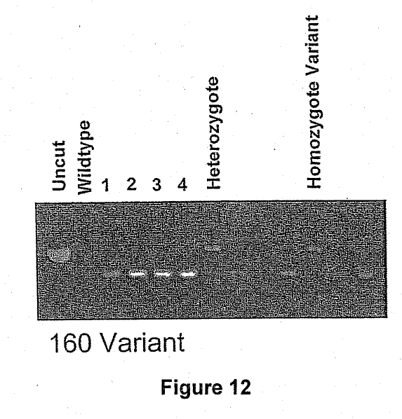

The R160H allele variant was tested for using a Sac II digest. Primers were used to amplify a target region of DNA in the MC1R gene. The amplification product was then subjected to a Sac II digest. Lanes 1 to 4 of the gel (see FIG. 12) show the results for the three tested subjects and the control sample. All four of these samples were homozygote wildtype. The size of the amplification product indicative of a homozygote wildtype for this allelic site is 436 bp, which after SAC II digestion yields a product of 327 bp. In homozygote variants no digestion occurs and so all the PCR amplification product remains at a size of 436 bp.

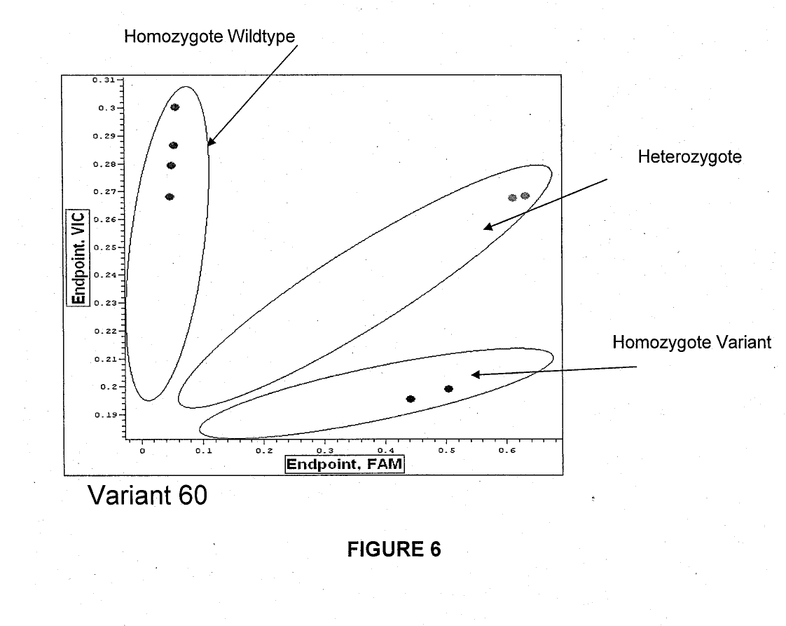

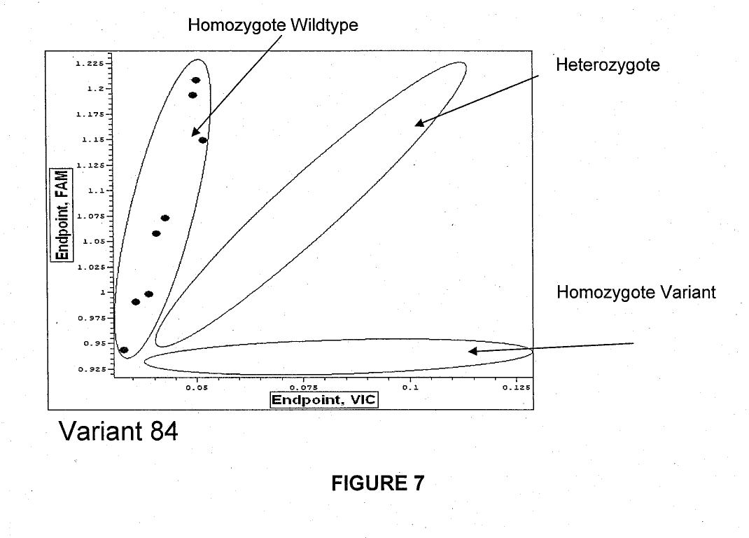

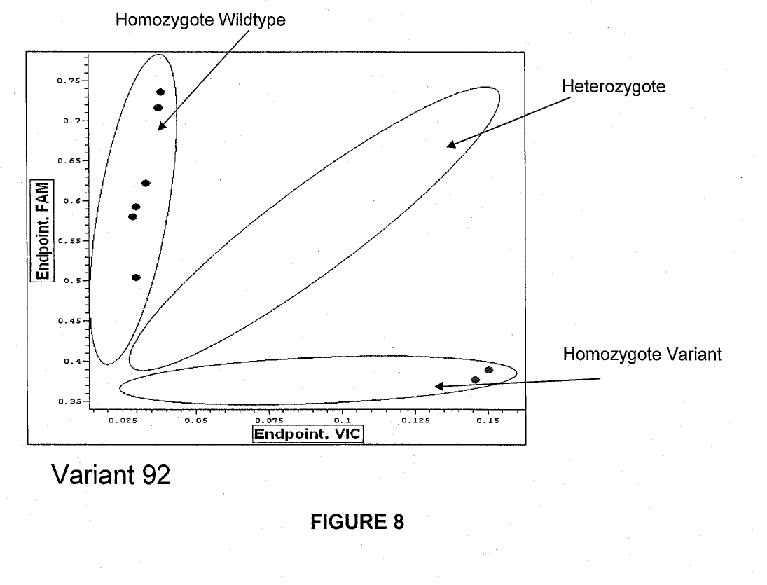

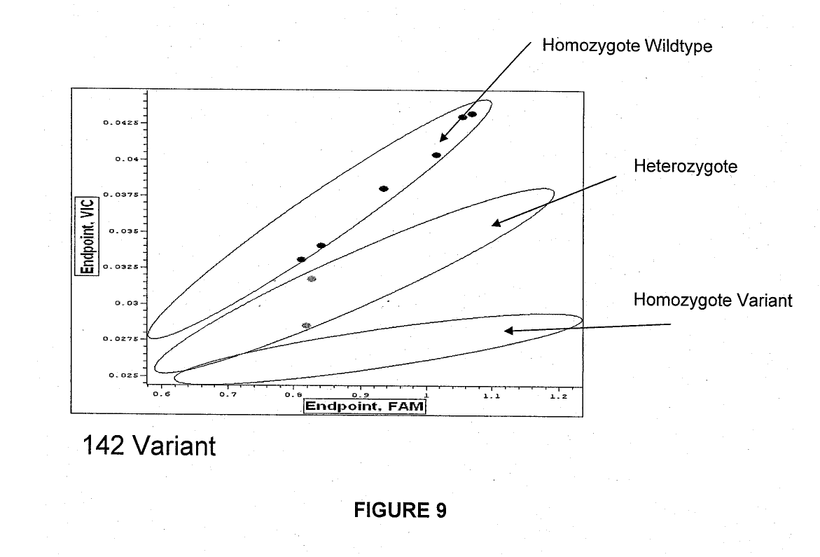

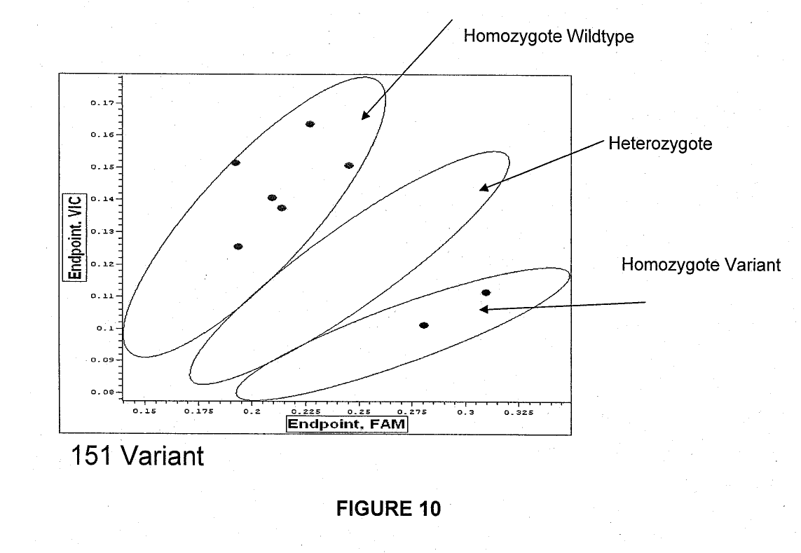

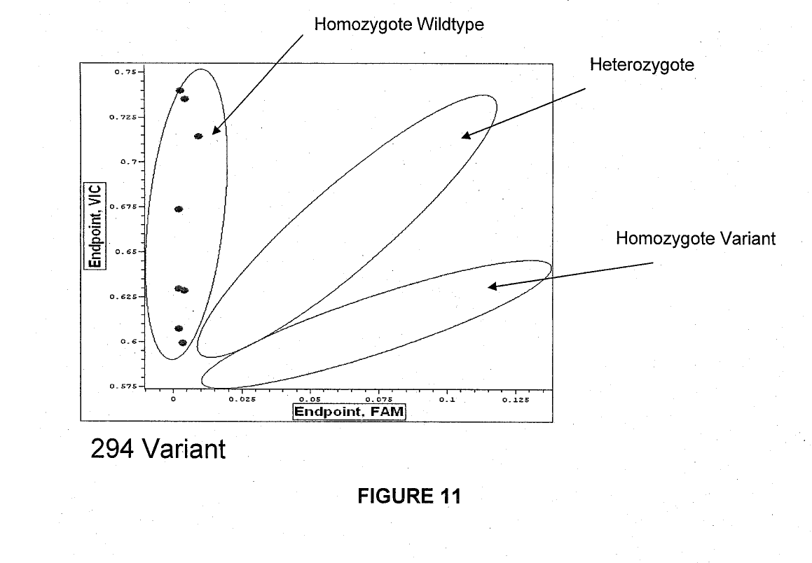

The results of the tests are shown in FIGS. 6 to 12. Two tests were run for each individual, and a control sample was also tested twice. The control sample genotype was a heterozygote for V60L, homozygote variant for V92M and wildtype for the remaining alleles. The first individual tested was determined to have Type 3 skin (no MC1R variants found). The second individual tested was determined to have Type 3 skin (no MC1R variants found). The third individual tested was determined to have Type 2 skin (homozygote for variants V60L and R151C and heterozygote for R142H).

It is clear that the material obtained through this collection method is sufficient for MC1R genotyping. Specifically, the collection of DNA samples via the buccal swab collection method yields sufficient DNA for use in an assay of this kind.

### Example 6

### Analysis of the Effectiveness of Sunscreen Regimes

Background:

UVB is the spectrum of UVR that causes erythema (sunburn), the visual cue that overexposure to UV has occurred. The frequency and severity of erythema is often used as a measure of success of an individual's sun protection habits. However, it is now known that UVA contributes to skin and DNA damage but it was not until recently that sunscreens and sunblocks contained agents directed at blocking UVA. Thus a scenario is created whereby an individual using sunscreen was protecting themselves against the erythema inducing effects of UVB but perhaps suffering even more prolonged exposure than they would have otherwise to the mutagenizing effects of UVA. An individual in this situation would self-report that they use sunscreens and a health care provider would mistakenly assess that measures were being taken to prevent UVR damage when in fact the patient was not being protected from UVA and ultimately risks could still be very high.

As carried out in Example 1, measuring the level of the 3895 bp mitochondrial DNA deletion in the epidermis collected with a cotton swab at regular intervals such as biweekly or monthly can provide health care providers with a real-time, quantitative monitoring tool to compare against treatment recommendations to determine their effectiveness in preventing skin damage caused by UVR.

This collection methodology permits regular sampling without any side effect or discomfort. The swab yields extremely low levels of nucleic acids (approximately 0.1 ng) which would not be useful for an assay targeting nuclear DNA however mitochondrial DNA targets such as the 3895 bp deletion are in much greater abundance (approximately 1000 fold greater) and are uniquely suited to a collection method with such extremely low yields.

Study:

The utility of mtDNA deletion analysis in identifying poor sun habits or ineffective protection methods is demonstrated by the results of two individuals of similar age and skin tone. Patient 202 did not use sunscreens at all while Patient 225 used sunscreens effectively. Their levels of damage reflect this behaviour with Patient 202 having approximately a 10 fold greater quantity of the 3895 bp deletion indicating high levels of DNA damage from UVR exposure than Patient 225 (where a 3 CT difference is equivalent to a 10 fold difference in target quantity). Though phenotypes of these two individuals indicate that risk for UVR associated skin damage would be similar or equivalent, the 3895 deletion coupled with the non-invasive swab collection is able to demonstrate that Patient 202 is at greater risk, presumably due to behavioural differences such as sunscreen use.

TABLE 5
Patient Profile and Risk Factors for UVR Damage

| | | | | | | Effective |
|---|---|---|---|---|---|---|
| | CT (3895 bp | | | | | Sunscreen |
| PATIENT | deletion) | AGE | SEX | Hair | Skin | use |
| 202 | 23.16 | 25 | Male | Black | Dark | No |
| | | | | | Brown | |
| 225 | 26.06 | 28 | Female | Brown | Light | Yes |
| | | | | | Beige | |

### Example 7

### Analysis of MC1R Variants and mtDNA Deletion to Predict Effectiveness of Sun Care Regimes

The combined utility of monitoring both the DNA damage with the UVR associated 3895 biomarker as a proxy for estimating the effectiveness of sun care regimes, as well as the determination of MC1R variant status is demonstrated below where two patients having similar age, skin colour, and sunscreen use have different levels of DNA damage presumably as a byproduct of different MC1R genotypes. Patient 300 has no variants associated with an increased susceptibility to the damaging effects of UVR on the skin while Patient 303 has one variant at amino acid position 92. Even though phenotypically the patients are very similar and would score similarly on the Fitzpatrick phototype scale (Type 3) the increased susceptibility is reflected in a 3 fold greater level of DNA damage in the Patient having the MC1R variant than in the patient without (where 1 CT difference is approximately 3 fold difference in target quantity).

TABLE 6
Comparative Analysis of MC1R Variance and mtDNA Deletion for Two Patients

| | CT of | | | | | | | | | | | | Effective |
|---|---|---|---|---|---|---|---|---|---|---|---|---|---|
| | 3895 bp | MC1R Variant (Amino Acid Position) | | | | | | | | | | Skin | Use of |
| PATIENT | deletion | 60 | 84 | 92 | 142 | 151 | 160 | 294 | AGE | SEX | Hair | Tone | sunscreens |
| 303 | 26.1 | 0 | 0 | 1 | 0 | 0 | 0 | 0 | 42 | Female | Black | Olive | Yes |
| 300 | 27.11 | 0 | 0 | 0 | 0 | 0 | 0 | 0 | 45 | Female | Brown | Olive | Yes |

As previously discussed, skin damage caused my UVR is a multifactorial process and, accordingly, it cannot be assumed that an individual with MC1R variants will always have higher damage than an individual without, nor will it be the case that an individual who reports regular sunscreen use will necessarily have lower damage than one who reports no sunscreen use. Combining these two tests provide the health care provider with the insight they require to make more informed recommendations to those in their care.

The following two tables illustrate this point as individuals with variants and without are generally equally represented across the DNA damage spectrum.

TABLES 7A & 7B
Comparison of MC1R Variance to Level of DNA Damage
A.

| | % of | % of |
|---|---|---|
| | population | population |
| | with | without |
| | variants | variants |
| low damage | 30 | 70 |
| average damage | 37 | 63 |
| high damage | 44 | 58 |

B.

| | | | | | | | | | | | | | # of |
|---|---|---|---|---|---|---|---|---|---|---|---|---|---|
| Damage | 60 | 84 | 92 | 142 | 151 | 160 | 294 | SUM | NO V | ONE V | TWO V | >0 V | patients |
| Low | 0 | 0 | 1 | 1 | 8 | 8 | 1 | 19 | 6 | 9 | 5 | 14 | 20 |
| Avg | 10 | 1 | 18 | 3 | 16 | 16 | 4 | 68 | 28 | 28 | 20 | 48 | 76 |
| High | 2 | 1 | 2 | 0 | 2 | 5 | 0 | 12 | 7 | 6 | 3 | 9 | 16 |

NO V Patients with no variants
ONE V Patients with one heterozygous variant in v60, v80, or v92
TWO V Patients with two het variants or one homo variant in v60, v80, and/or v92
>0 V Patients with at least one variant in v60, v80, or v92
Low Low damage group; C(t) is one standard deviation above population mean
Avg + High Average and above damage group; C(t) is less than one standard deviation above population mean

An exception is observed in the next two tables when considering only variants not typically associated with an at risk phenotype. These variants at amino acid positions 60 and 92, are under-represented in the low damage population perhaps indicating that their unique combination of susceptibility to damage as a result of MC1R variants coupled with a lack of visual cues such as fair skin or red hair, indicating increased risk result in a consistently higher level of damage in carriers.

TABLE 8
Comparison of MC1R v60 and v92 Variants to Level of DNA Damage
Pooled Proportion Tests Using Only v60 and v92

| Damage | NO V | >0 V | # of patients |
|---|---|---|---|
| LOW | 19 | 1 | 20 |
| Avg + High | 63 | 29 | 92 |

| | | | | | | | | | | delta | | phat(1- | | | sig @ | sig @ |
|---|---|---|---|---|---|---|---|---|---|---|---|---|---|---|---|---|
| | | n1 | n2 | p1 | p2 | n1 + n2 | 1/n1 + 1/n2 | p1hat | p2hat | phat | phat | phat) | z | p | .95? | .90? |
| low vs | no v | 20 | 92 | 19 | 63 | 112 | 0.06 | 0.95 | 0.68 | 0.27 | 0.73 | 0.20 | 2.43 | 0.02 | YES | YES |
| avg + high | >0 v | 20 | 92 | 1′ | 29 | 112 | 0.06 | 0.05 | 0.32 | 0.27 | 0.27 | 0.20 | 2.43 | 0.02 | YES | YES |

no v Patients with no variants
one v Patients with one heterozygous variant in v60, v80, or v92
two v Patients with two het variants or one homo variant in v60, v80, and/or v92
>0 v Patients with at least one variant in v60, v80, or v92
Low Low damage group; C(t) is one standard deviation above population mean
Avg + High Average and above damage group; C(t) is less than one standard deviation above population mean

### Example 8

### Needle Sampling for Determination of PhotoAge

An additional tool that makes use of a minimally invasive sampling procedure coupled with a mitochondrial marker for the purpose of evaluating UVR exposure is provided for by sampling the skin tissue on the face or head such as the earlobe or near the brow ridge with a small gauge needle (28 gauge), expelling the micro-core into solution, performing nucleic acid extraction and measuring the amount of a mitochondrial target associated with UVR damage such as the 3895 bp deletion (see Example 1). This result is then compared against a database of results and classified into an age cohort based upon the level of 3895 bp deletion. The database is created by measuring 3895 bp deletion levels in multiple individuals in 5 year intervals from ages 0 to 80 years, finding clusters or means for each interval, removing outliers, and correlating the range of level of 3895 bp deletion with an age range. For example, CT values from 21.5-23.5 may correlate with an age range of 45-65 years of age. The results of an individual's test would be then compared against their chronological age to determine if they have higher or lower photodamage than other individuals in their age group, and assign them a photoage based on their result.

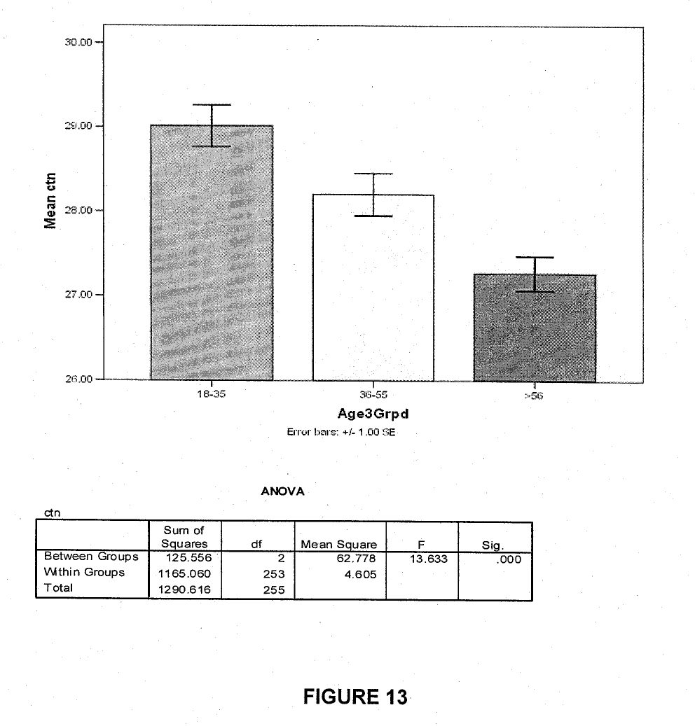

To create this database, microcores were collected from 289 individuals at both their earlobes and brow ridges. As shown in FIG. 13, following DNA extraction and quantification of the level of the 3895 bp deletion the database was constructed as described above with age cohorts falling into 3 groups having a significant difference between them (p<0.01).

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A diagnostic method for determining the skin state and genetic predisposition of a subject to UVR damage, comprising:
(a) collecting tissue samples from a subject;
(b) assaying a first skin sample for mitochondrial DNA (mtDNA) aberrations;
(c) assaying a second skin sample for one or more melanocortin 1 receptor (MC1R) variants; and
(d) determining the skin state and genetic predisposition of the subject to UVR damage based on the detection of the mtDNA aberrations and MC1R variant(s) in the skin samples.

2. The method of claim 1 wherein the aberration is selected from the group consisting of deletions, substitutions, and insertions.

3. The method of claim 2 wherein the aberration is an mtDNA deletion.

4. The method of claim 3 wherein the deletion is a 3895 by mtDNA deletion between nucleic acids 546 to 4444 of the mtDNA genome.

5. The method of claim 1 wherein the one or more MC1R variants are selected from the group consisting of D84E, R142H, R151C, R160H, D294H, V60L, and V92M.

6. The method of claim 1 wherein at least the first tissue sample is a skin sample obtained using a non-invasive or minimally invasive skin collecting technique.

7. The method of claim 6 wherein the skin sample is collected using a Sterile swab, cotton tip swap, a small gauge needle to collect micro-cores of skin tissue, or a combination thereof.

8. The method of claim 7 wherein the skin is collected from the dermal or epidermal layer of the subject.

9. The method of claim 8 wherein the skin sample is derived from the heel, nose, inner arm, ear, mouth, scalp, chest, shoulder, buttock, back, face, nape of the neck, hand, head, or a combination thereof.

10. Use of the method of claim 1 for predicting photoaging, UVR damage or skin disease.

11. Use of the method of claim 1 for determining a prophylactic or therapeutic treatment for preventing or ameliorating photoaging, UVR damage or skin cancer.

12. A non-invasive method for monitoring photoaging, UVR damage or skin disease, comprising:
(a) collecting a skin sample from a subject using a non-invasive skin sampling technique;
(b) assaying the skin sample for mitochondrial DNA (mtDNA) aberrations at regular intervals over a prescribed period of time; and
(c) determining any changes in mtDNA aberration identified over the prescribed period of time.

13. The method of claim 12 wherein the non-invasive skin collecting technique yields ultra low levels of DNA.

14. The method of claim 13 wherein said ultra low levels of DNA is about 0.1 ng of nucleic acid.

15. A method for monitoring a subject's response to a preventative or therapeutic treatment for photoaging, UVR damage or skin disease, comprising:
(a) collecting a first skin sample from a subject;
(b) assaying the first skin sample for mitochondrial DNA (mtDNA) aberrations;
(c) assaying the first skin sample for one or more melanocortin 1 receptor (MC1R) variants;
(d) determining the skin state and genetic predisposition of the subject to UVR damage based on the detection of the mtDNA aberrations and MC1R variant(s) in the first skin sample;
(e) providing a preventative or therapeutic treatment for photoaging, UVR damage or skin disease;
(f) collecting a second skin sample from a subject;
(g) assaying the second skin sample for mitochondrial DNA (mtDNA) aberrations;
(h) repeating steps (f) and (g) at regular intervals over a prescribed period of time; and
(i) comparing the level of mtDNA aberrations between the first skin sample and the skin samples taken at regular intervals to detect changes in mtDNA aberrations, thereby monitoring the effectiveness of the treatment; and
(j) optionally, adjusting the treatment based on the genetic predisposition of the subject and the detected changes in mtDNA aberrations.

16. The method of claim 15 wherein the first skin sample, second skin sample and skin samples taken at regular intervals are obtained using a non-invasive or minimally invasive skin collecting technique.

17. The method of claim 16 wherein the non-invasive or minimally invasive skin collecting technique used to collect the second skin sample and samples taken at regular intervals yields ultra low levels of DNA.

18. The method of claim 17 wherein said ultra low levels of DNA is about 0.1 ng of nucleic acid.

19. The method of claim 15 wherein said regular intervals are biweekly or monthly.

20. A method of screening for an effective therapeutic or cosmeceutic agent for the treatment of photoaging, UVR damage or skin disease, comprising;
(a) collecting a first skin sample from a subject;
(b) assaying the first skin sample for mitochondrial DNA (mtDNA) aberrations;
(c) treating the subject with the therapeutic or cosmeceutic agent;
(d) collecting a second skin sample from a subject following a prescribed period of time;
(e) assaying the second skin sample for mitochondrial DNA (mtDNA) aberrations; and
(f) comparing the level of mtDNA aberrations between the first skin sample and the second skin sample against a control to determine the effectiveness of the therapeutic or comesceutical agent.

21. The method of claim 20 wherein the first skin sample and second skin sample are obtained using a non-invasive or minimally invasive skin collecting technique.

22. A method for determining the level of photodamage of a subject, comprising:
(a) collecting a skin sample from a subject;
(b) assaying the skin sample for mitochondrial DNA (mtDNA) deletions;
(c) comparing the level of mtDNA deletions of the skin sample against a population of mtDNA deletions categorized according to age co-horts, and assigning a photoage to the subject; and
(d) determining the level of photodamage of the subject by comparing the subject's chronological age to the assigned photoage.

23. The method of claim 22 where the mitochondrial DNA deletion is the 3895 bp deletion.

24. A diagnostic kit for determining the skin state and genetic predisposition of a subject to UVR damage, comprising:
(a) material for collecting tissue samples; and
(b) suitable primers, probes and reagents for carrying out MC1R genotyping and the detection of mtDNA aberrations.


SEQUENCE LISTING (12 sequences)

SEQ ID NO: 1 - DNA, 12674 bp, Homo sapiens
gatcacaggtctatcaccctattaaccactcacgggagctctccatgcatttggtattttcgtctggggggtatgcacgcgatagcattgcgagacgctggagccggagcaccctatgtcgcagtatctgtctttgattcctgcctcatcctattatttatcgcacctacgttcaatattacaggcgaacatacttactaaagtgtgttaattaattaatgcttgtaggacataataataacaattgaatgtctgcacagccactttccacacagacatcataacaaaaaatttccaccaaaccccccctcccccgcttctggccacagcacttaaacacatctctgccaaaccccaaaaacaaagaaccctaacaccagcctaaccagatttcaaattttatcttttggcggtatgcacttttaacagtcaccccccaactaacacattattttcccctcccactcccatactactaatctcatcaatacaacccccgcccatcctacccagcacacacacaccgctgctaaccccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgaaccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggtcataaagcctaaatagcccacacgttccccttaaataagacatcacgatg

SEQ ID NO: 2 - DNA, 3895 bp, Homo sapiens
  misc_feature (2559)..(2559): n is a, c, g, or t
ccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaaggaacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctancttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaa

SEQ ID NO: 3 - DNA, 2360 bp, Homo sapiens
gagagggcaggtcccggggaagctccggactcctagaggggcggccaggtgggggccctggtgaccaggacagactgtggtgttttttaacgtaaaggagatccgcggtgtgagggaccccctgggtcctgcacgccgcctggtggcaggccgggccatggtgggtgctcacgcccccggcatgtggccgccctcagtgggaggggctctgagaacgactttttaaaacgcagagaaaagctccattcttcccaggacctcagcgcagccctggcccaggaaggcaggagacagaggccaggacggtccagaggtgtcgaaatgtcctggggacctgagcagcagccaccagggaagaggcagggagggagctgaggaccaggcttggttgtgagaatccctgagcccaggcggtagatgccaggaggtgtctggactggctgggccatgcctgggctgacctgtccagccagggagagggtgtgagggcagatctgggggtgcccagatggaaggaggcaggcatgggggacacccaaggccccctggcagcaccatgaactaagcaggacacctggaggggaagaactgtggggacctggaggcctccaacgactccttcctgcttcctggacaggactatggctgtgcagggatcccagagaagacttctgggctccctcaactccacccccacagccatcccccagctggggctggctgccaaccagacaggagcccggtgcctggaggtgtccatctctgacgggctcttcctcagcctggggctggtgagcttggtggagaacgcgctggtggtggccaccatcgccaagaaccggaacctgcactcacccatgtactgcttcatctgctgcctggccttgtcggacctgctggtgagcgggagcaacgtgctggagacggccgtcatcctcctgctggaggccggtgcactggtggcccgggctgcggtgctgcagcagctggacaatgtcattgacgtgatcacctgcagctccatgctgtccagcctctgcttcctgggcgccatcgccgtggaccgctacatctccatcttctacgcactgcgctaccacagcatcgtgaccctgccgcgggcgcggcgagccgttgcggccatctgggtggccagtgtcgtcttcagcacgctcttcatcgcctactacgaccacgtggccgtcctgctgtgcctcgtggtcttcttcctggctatgctggtgctcatggccgtgctgtacgtccacatgctggcccgggcctgccagcacgcccagggcatcgcccggctccacaagaggcagcgcccggtccaccagggctttggccttaaaggcgctgtcaccctcaccatcctgctgggcattttcttcctctgctggggccccttcttcctgcatctcacactcatcgtcctctgccccgagcaccccacgtgcggctgcatcttcaagaacttcaacctctttctcgccctcatcatctgcaatgccatcatcgaccccctcatctacgccttccacagccaggagctccgcaggacgctcaaggaggtgctgacatgctcctggtgagcgcggtgcacgcggctttaagtgtgctgggcagagggaggtggtgatattgtgtggtctggttcctgtgtgaccctgggcagttccttacctccctggtccccgtttgtcaaagaggatggactaaatgatctctgaaagtgttgaagcgcggacccttctgggtccagggaggggtccctgcaaaactccaggcaggacttctcaccagcagtcgtggggaacggaggaggacatggggaggttgtggggcctcaggctccgggcaccaggggccaacctcaggctcctaaagagacattttccgcccactcctgggacactccgtctgctccaatgactgagcagcatccaccccaccccatctttgctgccagctctcaggaccgtgccctcgtcagctgggatgtgaagtctctgggtggaagtgtgtgccaagagctactcccacagcagccccaggagaaggggctttgtgaccagaaagcttcatccacagccttgcagcggctcctgcaaaaggaggtgaaatccctgcctcaggccaagggaccaggtttgcaggagcccccctagtggtatggggctgagccctcctgagggccggttctaaggctcagactgggcactggggcctcagcctgctttcctgcagcagtcgcccaagcagacagccctggcaaatgcctgactcagtgaccagtgcctgtgagcatggggccaggaaagtctggtaataaatgtgactcagcatcacccacctta

SEQ ID NO: 4 - RNA, 3115 nt, Homo sapiens
agacgcagucuucagcaaggaagugcugggaacgcccuggagugaacccaggaagaugccugcagugggugccagggccccucuccaccgucccugcugggcuucggggccacgcccgacugcugugaacggccugcggagcaccacgugcgacggcuggaggcgagaggucugccuuugauguggcuguuggugcagggccuguggugccuuccgcagcggaaauggcgcgccgcccggggagggcgggagcagcgucccgggugccccugugaggaugagcgacgagaugacuggagggucccugaagaccucacuagggugcccccagccgguccgcucccaggaagcgacacccccacagccccagggcugcagcugagggggucgccacucuggcugggcgaggcugggcccuugggggcaggcgccagaguggccucaggcucuacaagaugccugaaaacaccaaccucuccagggcucacuagcauuggacgcuuucacgcucugcccuggccggaagcccccucaccccgcgcgaugugcaaacuccugcagggcucacucaguuuccagaacuuuaauuauuggaaaguucucccugguccagcccccaaaucugccgugaacguugacagcugaguugcugcuccaugcgugcuuuggcugagagcagaggggaccccuguccucccugagcugcugacgaggggaggggugaaggguggggccucuggagagggcaggucccggggaagcuccggacuccuagaggggcggccaggugggggcccuggugaccaggacagacugugguguuuuuuaacguaaaggagauccgcggugugagggacccccuggguccugcacgccgccugguggcaggccgggccauggugggugcucacgcccccggcauguggccgcccucagugggaggggcucugagaacgacuuuuuaaaacgcagagaaaagcuccauucuucccaggaccucagcgcagcccuggcccaggaaggcaggagacagaggccaggacgguccagaggugucgaaauguccuggggaccugagcagcagccaccagggaagaggcagggagggagcugaggaccaggcuugguugugagaaucccugagcccaggcgguagaugccaggaggugucuggacuggcugggccaugccugggcugaccuguccagccagggagagggugugagggcagaucugggggugcccagauggaaggaggcaggcaugggggacacccaaggcccccuggcagcaccaugaacuaagcaggacaccuggaggggaagaacuguggggaccuggaggccuccaacgacuccuuccugcuuccuggacaggacuauggcugugcagggaucccagagaagacuucugggcucccucaacuccacccccacagccaucccccagcuggggcuggcugccaaccagacaggagcccggugccuggagguguccaucucugacgggcucuuccucagccuggggcuggugagcuugguggagaacgcgcuggugguggccaccaucgccaagaaccggaaccugcacucacccauguacugcuucaucugcugccuggccuugucggaccugcuggugagcgggagcaacgugcuggagacggccgucauccuccugcuggaggccggugcacugguggcccgggcugcggugcugcagcagcuggacaaugucauugacgugaucaccugcagcuccaugcuguccagccucugcuuccugggcgccaucgccguggaccgcuacaucuccaucuucuacgcacugcgcuaccacagcaucgugacccugccgcgggcgcggcgagccguugcggccaucuggguggccagugucgucuucagcacgcucuucaucgccuacuacgaccacguggccguccugcugugccucguggucuucuuccuggcuaugcuggugcucauggccgugcuguacguccacaugcuggcccgggccugccagcacgcccagggcaucgcccggcuccacaagaggcagcgcccgguccaccagggcuuuggccuuaaaggcgcugucacccucaccauccugcugggcauuuucuuccucugcuggggccccuucuuccugcaucucacacucaucguccucugccccgagcaccccacgugcggcugcaucuucaagaacuucaaccucuuucucgcccucaucaucugcaaugccaucaucgacccccucaucuacgccuuccacagccaggagcuccgcaggacgcucaaggaggugcugacaugcuccuggugagcgcggugcacgcggcuuuaagugugcugggcagagggagguggugauauuguguggucugguuccugugugacccugggcaguuccuuaccucccugguccccguuugucaaagaggauggacuaaaugaucucugaaaguguugaagcgcggacccuucuggguccagggaggggucccugcaaaacuccaggcaggacuucucaccagcagucguggggaacggaggaggacauggggagguuguggggccucaggcuccgggcaccaggggccaaccucaggcuccuaaagagacauuuuccgcccacuccugggacacuccgucugcuccaaugacugagcagcauccaccccaccccaucuuugcugccagcucucaggaccgugcccucgucagcugggaugugaagucucuggguggaagugugugccaagagcuacucccacagcagccccaggagaaggggcuuugugaccagaaagcuucauccacagccuugcagcggcuccugcaaaaggaggugaaaucccugccucaggccaagggaccagguuugcaggagccccccuagugguauggggcugagcccuccugagggccgguucuaaggcucagacugggcacuggggccucagccugcuuuccugcagcagucgcccaagcagacagcccuggcaaaugccugacucagugaccagugccugugagcauggggccaggaaagucugguaauaaaugugacucagcaucacccaccuuaaaaaaaaaaaaaaaaa

SEQ ID NO: 5 - DNA, 28 bp, Artificial Sequence
  3895 mtDNA deletion forward primer
ctgctaaccccataccccgaaaatgttg

SEQ ID NO: 6 - DNA, 31 bp, Artificial Sequence
  3895 mtDNA deletion reverse primer
gaaggattatggatgcggttgcttgcgtgag

SEQ ID NO: 7 - DNA, 21 bp, Artificial Sequence
  mtDNA genome forward primer
cgttccagtgagttcaccctc

SEQ ID NO: 8 - DNA, 23 bp, Artificial Sequence
  mtDNA genome reverse primer
cactctttacgccggcttctatt

SEQ ID NO: 9 - DNA, 20 bp, Artificial Sequence
  MC1R-294 forward primer
cgccctcatcatctgcaatg

SEQ ID NO: 10 - DNA, 19 bp, Artificial Sequence
  MC1R-294 reverse primer
ggctgtggaaggcgtagat

SEQ ID NO: 11 - DNA, 16 bp, Artificial Sequence
  MC1R-294V1 VIC probe
ccatcatcgaccccct

SEQ ID NO: 12 - DNA, 16 bp, Artificial Sequence
  MC1R-294M1 FAM probe
ccatcatccaccccct